(19) United States
(12) Patent Application Publication
(10) Pub. No.: US 2005/0175512 A1
(43) Pub. Date: Aug. 11, 2005

(54) Electro-optical air sterilizer with ionizer

(76) Inventor: Se Kit Yuen, Hong Kong (CN)

Correspondence Address: ARTHUR KING MA, 1030 CORONADO DRIVE, ARCADIA, CA, 91007, US

(21) Appl. No.: 10/964,262

(22) Filed: Oct. 12, 2004

(30) Foreign Application Priority Data
Feb. 10, 2004 (CN) 200410039128.5
Feb. 10, 2004 (CN) 200420002592.2

(51) Int. Cl. A61L 9/20

(52) U.S. Cl. 422/121

(57) ABSTRACT

This invention relates to an air purifier that uses both transportation vehicles battery and conventional indoor power supply, more particularly to an air purifier which employ UV tube to generate UV with wavelength of 253.7 nanometers, electronic circuit system is characterized by a high negative voltage output volume as stated, i.e. within the range of 4.5 kv and 8.5 kv which is able to kill bacteria, virus and mildew in the air while emits negative ions into the environment. This air purifier draws indoor air in and out of the air purifier, kills bacteria, viruses and moulds in the air and discharges electricity by means of negative high voltage output through carbon fibers to produce anions, so as to improve the quality of the air.

### BACKGROUND OF THE PRESENT INVENTION

1. Field of Invention

The present invention is related to an air sterilizer and more particularly it relates to a three-in-one air sterilizer which employ UV tube to generate UV with wavelength of 253.7 nanometers, which is able to kill bacteria, virus and mildew in the air while emits negative ions into the environment.

2. Description of Related Arts

### SUMMARY OF THE PRESENT INVENTION

Numerous air filters have been provided in prior art. For example, U.S. Pat. No. 3,967,927, U.S. Pat. No. 4,210,429; U.S. Pat. No. 5,171,060 and U.S. Pat. No. 5,330,722 all are illustrative of such prior art. While these units may be suitable for the particular purpose to which they address, they would not be as suitable for the purposes of the present invention as heretofore described.

As disclosed in U.S. Pat. No. 3,967,927, an ultraviolet lamp fixture for purifying the air within a room by means of passing the air over a plurality of hot cathode or other commercially available ultraviolet ray tubes. The tubes are mounted vertically within a decoratively covered, easily movable, pole-mounted housing having a motorized fan, which moves air through an opening in the lower portion of the housing over the tubes for purification. The purified air stream is exhausted through the top portion of the housing and returned to the room. The interior of the housing has deflector vanes which function to create a turbulent airflow in the area of the tubes to insure that contacting the lamps purifies all of the air passing through the lamp fixture. The lamp fixture is provided with means for filtering the circulating air. In an alternate embodiment, the air passes through a dehumidifier prior to passing by the tubes.

Furthermore, U.S. Pat. No. 4,210,429, a room air sterilizer for quietly removing irritating or harmful impurities from the air circulating within the room. The sterilizer removes from room air, particles down to 0.3 microns in size with 99.9% efficiency. The air sterilizer comprises a somewhat elongated upright housing having an easily-removable back, a two-speed blower that is preferably AC operated and disposed at the bottom of the housing, a pair of vertically disposed ultraviolet lamps and associated means for powering the lamps including push button switch means, and preferably three separate filters including a pre-filter disposed at the inlet of the blower, a highly efficient main filter element vertically stacked over the blower and lamps and a charcoal filter disposed over the main filter element. The blower sucks the air in the bottom of the sterilizer through the pre-filter and up adjacent to the lamps to the main filter element and charcoal filter, and from there the purified air passes to a top baffle cover where the air is exited in preferably four directions from the sterilizer.

In U.S. Pat. No. 5,330,722 a germicidal air sterilizer for trapping and destroying airborne microorganisms is disclosed. The air sterilizer includes an ultraviolet radiation source and a juxtaposed filter medium. One ultraviolet radiation source and filter medium is fixed and the other is displaceable, so that at least an upstream side of the filter medium is systematically exposed to germicidal levels of radiation. In a first preferred embodiment, a fixed ultraviolet lamp irradiates a cylindrical air filter, which is rotated on its longitudinal axis in close proximity to the lamp, so that the upstream side of the filter is systematically irradiated. In a second preferred embodiment, a radiant lamp fixture is moved reciprocally across an upstream side of a planar filter, to systematically irradiate the filter. In a third preferred embodiment, a radiant lamp fixture is rotated about an axis, which is orthogonal to its longitudinal midpoint, so that a circular area of a planar filter is irradiated. The advantage is that microorganisms trapped on the filters are exposed to a lethal dose of radiation and the air sterilizer is consistently effective at destroying a significant percentage of airborne microorganisms suspended in the air passed through the filter.

In addition, most conventional air sterilizers can only separate unwanted airborne particles from air to achieve so-called purification for air. However, this is a well known fact that anions in the air are good for health. It would be beneficial should air sterilizers can produce anions while separating unwanted airborne particles from air allows. A main object of the present invention is to provide an air sterilizer has the ability providing capacity for both indoor power supply and automobile power supply.

A main object of the present invention is to provide an air sterilizer which employ UV tube to generate UV with wavelength of 253.7 nanometers, which is able to kill bacteria, virus and mildew in the air while emits negative ions into the environment.

Another object of the present invention is to provide an air sterilizer can produce anions while separating unwanted airborne particles from air flows.

Accordingly, in order to accomplish the above objects, the present invention provides an air sterilizer, which comprises:
an air ventilator,
an UV emission tube, and negative high voltage electrical carbonization fiber. When the air is drawn into said air sterilizer through its ventilator, it goes through two different constant circulating processes. First process is to exercise negative high voltage on carbonization fiber to discharge electricity, thus creates negative ions and then emanates the air through vent. Second process sets off UV to kill bacteria, virus and mildew. There is a one incoming air vent and one outgoing air vent. Said high voltage carbonization fiber is fixed inside of an outgoing air vent, while said UV emission tube is located within said incoming air vent. Said air ventilator is positioned inside of said air sterilizer, taking in regular air with bacteria, virus, and mildew to said air sterilizer. It drives the moving air through said air sterilizer, forcing air with bacteria, virus and mildew to contact said UV tube, so that UV could perish bacteria, virus and mildew first, and then it pushes the air through the purification process of negative ionization and finally emanates fresh air with negative ion to improve the indoor air quality.

### BRIEF DESCRIPTION OF THE DRAWINGS

FIG. 1 is a side view of the present invention.

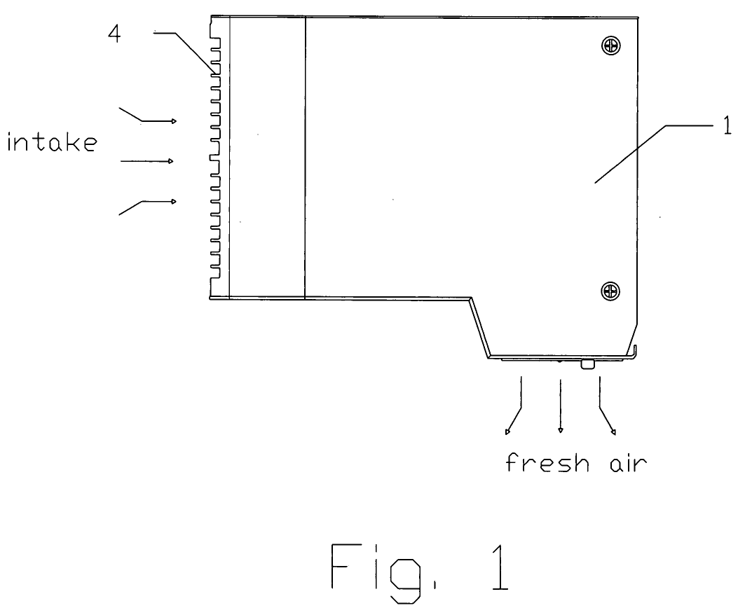

FIG. 2 is a cross-sectional view thereof;

FIG. 3 is another cross sectional view thereof;

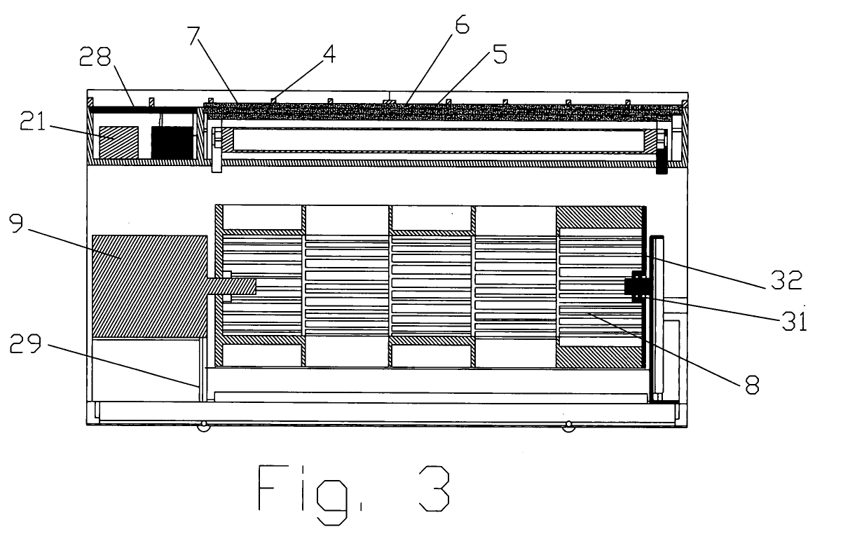

FIG. 4 is a front view thereof;

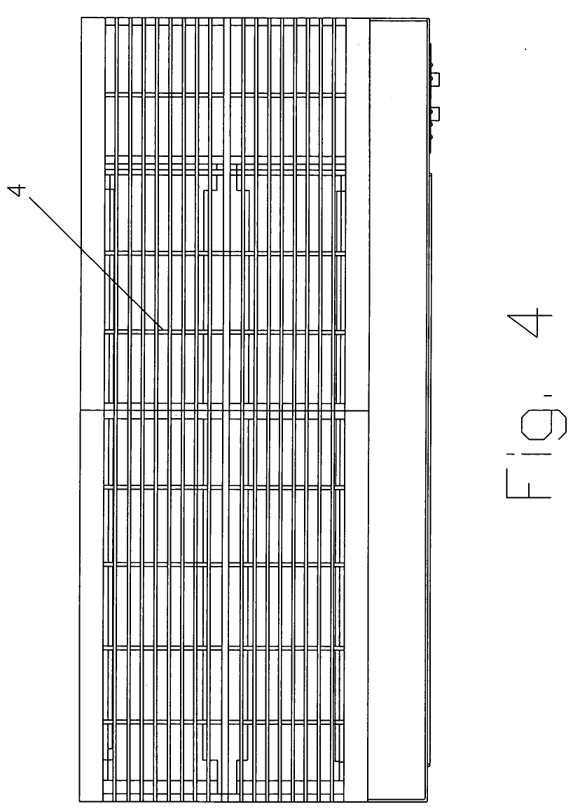

FIG. 5 is a rear view thereof;

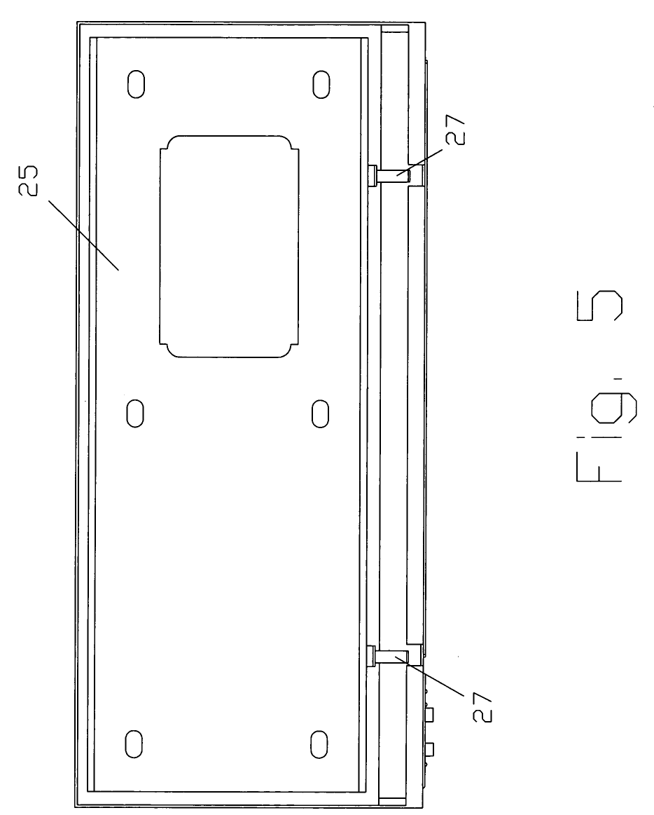

FIG. 6 is another side view thereof;

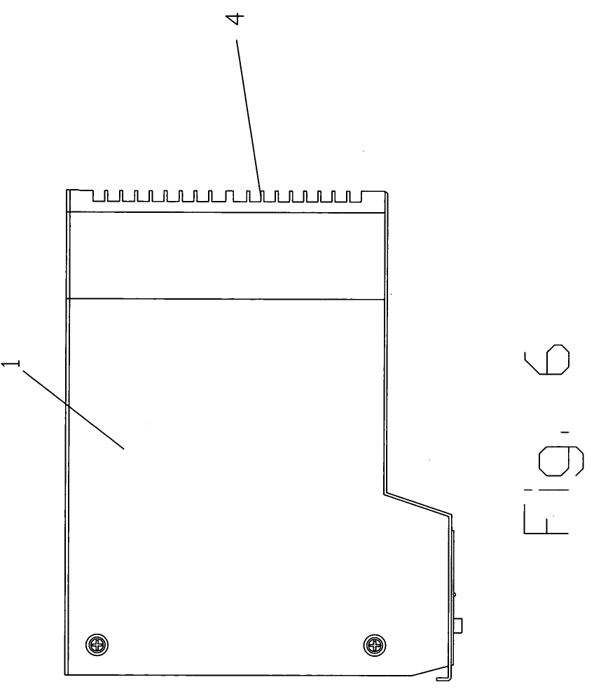

FIG. 7 is a supine view thereof;

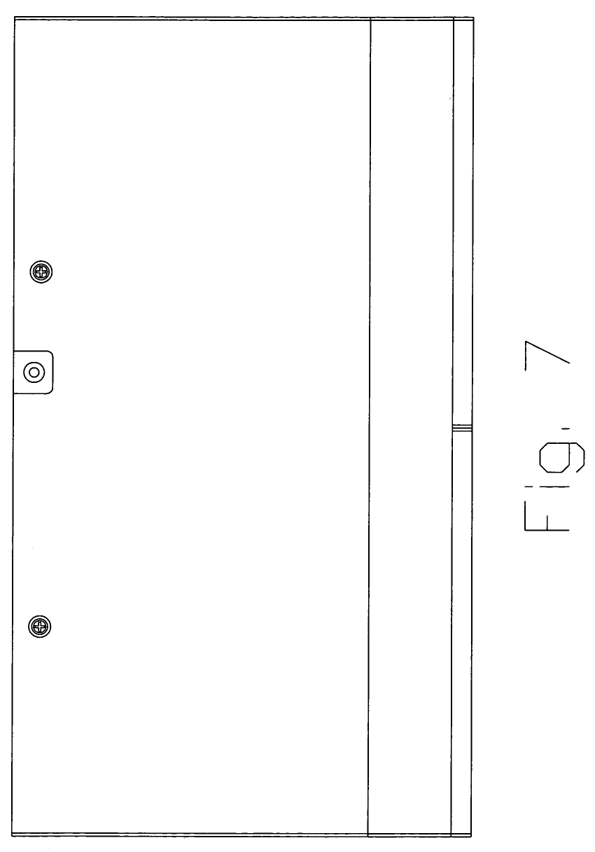

FIG. 8 is a base view thereof;

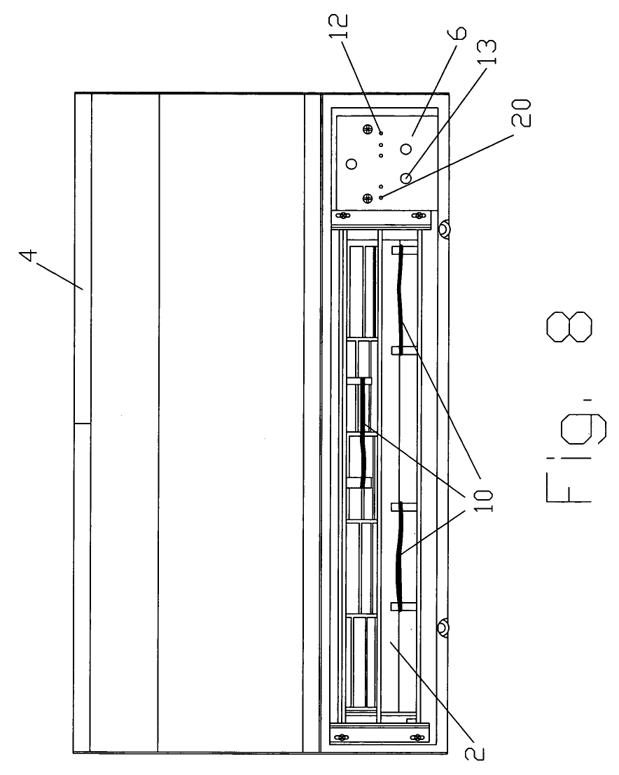

FIG. 9 is a diagram of components structure thereof.

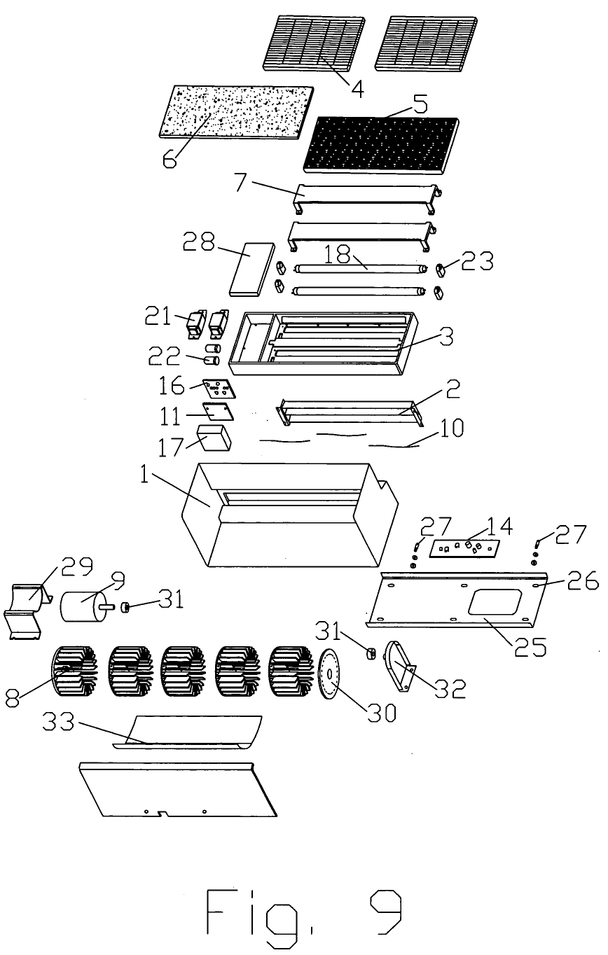

FIG. 10 displays a structure of UV emission tube.

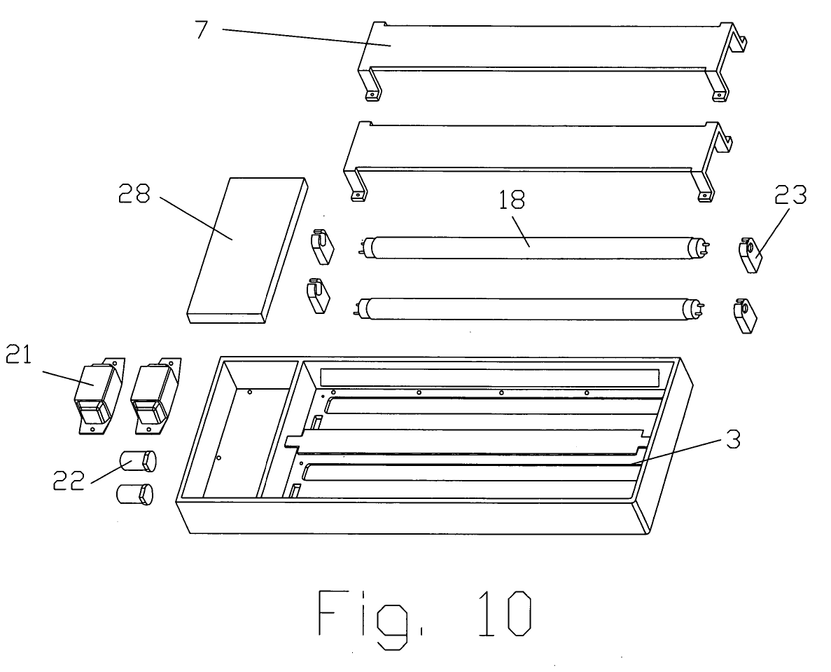

FIG. 11 displays a structure and a installation process of air ventilation blades.

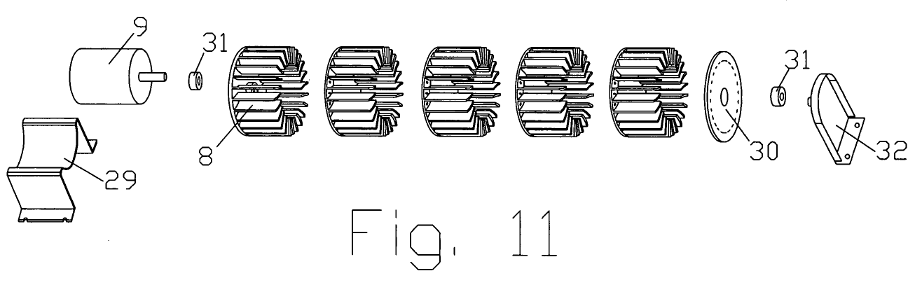

FIG. 12 displays a wall mounting installation process of said air sterilizer.

FIG. 13 displays installing said air sterilizer in a horizontal way.

FIG. 14 displays installing said air sterilizer in a vertical way.

### DETAILED DESCRIPTION OF THE PREFERRED EMBODIMENT

This three in one, electro-optical air sterilizer with ionizer has a cylinder shaped body (1). A rectangular outgoing air lattice (2) is located at the lower part of said body (1). A front of said body (1) has a shelf (3) for UV tube and incoming air lattice (4). Ionized air is released via outgoing air lattice (2).

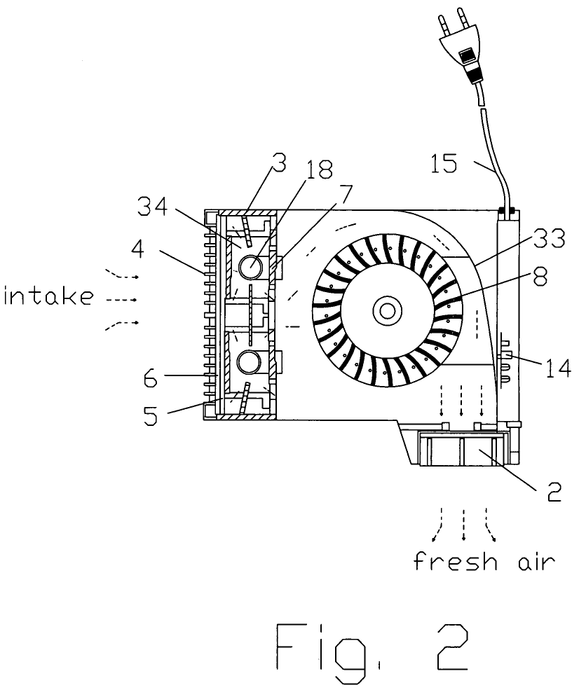

Detailed structural diagram of electro-optical air sterilizer with ionizer can be found in FIG. 2. A front part of said body (1) has a UV tube components shelf (3) and at last two incoming air lattices (4). While incoming air lattice (4) serve as air entrance, it also fasten an active carbon filter (5) and dust filtration screen (6) which closely attached in front of said active carbon filter (5). Right behind said active carbon filter (5) are two pieces of covered walls (7) and UV tube components shelf (3). There are at least five ventilators (8), one electrically operated motor (9), three sets of carbonization fiber (10) that are placed at an upper and lower parts of outgoing vent (2) respectively to ionize air.

Power electrical time control device (11), functional light indicator (12), on/off switch (13), electricity converter (14) and a power cord (15) are located at a lower part of said body (1).

At right lower portion of said body (1) has a function-controlling compartment (17) with cellular cover (16). Inside of said compartment (17) locates on/off switch (13), which is in charge of activating electrical operated motor (9), ventilator (8), UV tube (18) and carbonization fiber (10). There are many little round holes beside the on/off switch (13) to fasten different color light emission bipolar tubes (20) for light indications on various functions.

There is a power cord (15) on top of the body to connect supplied electricity from city. Front part of body's (1) interior locates a UV tube components shelf (3). There are at least two UV tubes (18), two light tube electron converters (21) two light tube activators (22), and four light tube seats (23). Protection cover (28) is located on top of an electron converter (21) and light tube activator (22). UV tube components shelf (3) is comprised with air collection wall (24), front and back cover walls (7) and UV tube. After removing incoming air lattice (4), dust filtration screen (6), active carbon filter (5) and the two cover walls (7) on the front, user will be able to replace the UV tube (18) inside of said body (1).

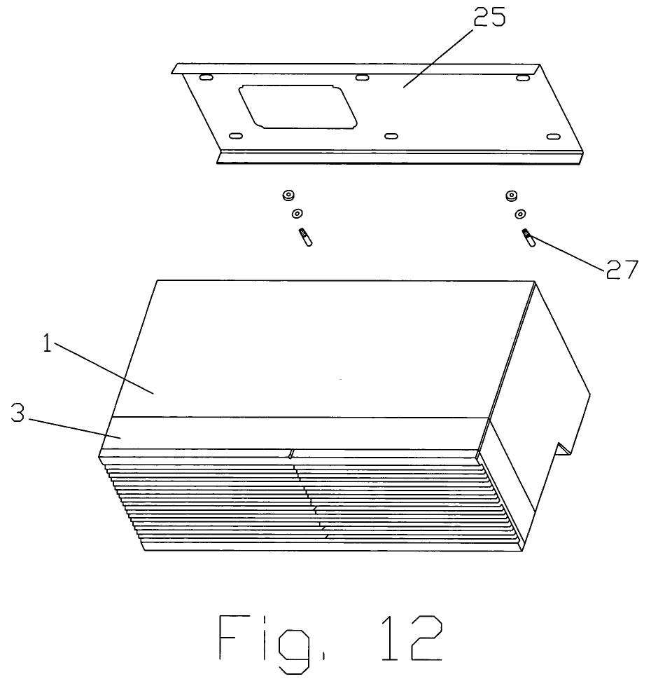

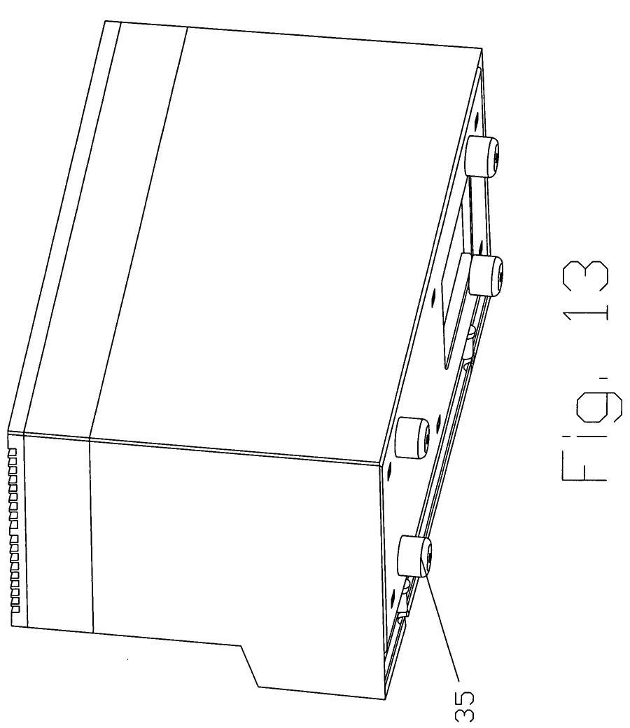

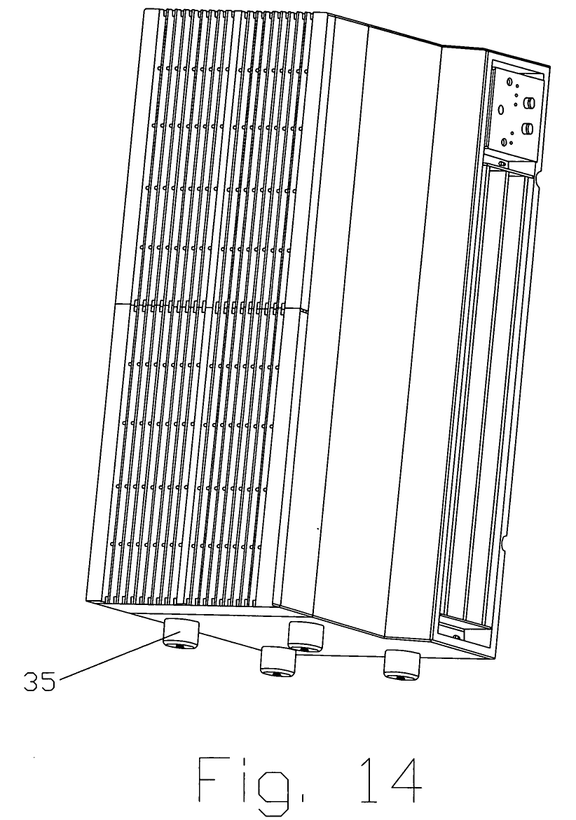

There is a wall-mounting bracket (25) at the back of said body (1). Said bracket (25) is equipped with wall mounting holes (26). When installing said body (1), first is to fasten said bracket (25) on a wall, then hang said body (1) on said wall mounting bracket (25), then use two screws to fasten said body (see FIG. 12). User can add shockproof feet (35) to place said body (1) vertically (see FIG. 14) or horizontally (see FIG. 13) to accommodate various conditions and locations.

Air ventilator parts inside of said body (1) include air directing board (33), motor supporting base (29), electrical operated motor (9), five fan blades (8), ventilator cover (30), two bearings (31), and a bearing supporting base (32).

In practice, when activating Photoelectric Sterilization Air Sterilizer, a ventilator (8) absorbs air with bacteria and mildews through active carbon filter (5), moves it into said UV tube components shelf (3). An air collection wall (24) inside of said UV tube components shelf (3) allows air to enter into collection wall (24) easily. UV tube (14) is placed in a center part of said air collection wall (24). When air that contains bacteria, viruses and mildews enter into an air-incoming vent (34) between UV tube (18) and air collection wall (24), UV released from UV tube (18) will perish bacteria, viruses and mildews in the air. Afterwards, the purified air will be sucked by a set of five ventilators (8) into a cover wall (7). It then passes through negative high voltage carbonization fiber (10), which is located on top and bottom of air lattices, to generate negative ions. Finally, the fresh air with negative ions is released from outgoing air lattice (2) to improve indoor air quality.

In front and back of said air collection wall (24) are cover walls (7). They are used to prevent excessive emission from UV tube, protect user's eyes and skin.

Circuitry Theory of Electro-Optical Air Sterilizer with Ionizer:

There are different paths for input power voltage when it enters into the appliance. One passes through on/off switch (13) to a circuitry that generates negative ion via high voltage, provides a negative high voltage output. The other after going through rectifier, it provides power via speed controlling circuitry, supply electricity to air ventilator, and go through alternate current (AC) circuitry to light tube electron converter (21) light tube activators (22), and a circuitry that activates UV tube (18) rectifier. Another path of input power goes through direct current (DC) toward the circuitry of negative ion generator, provides electricity to auto circulating controlling circuitry that activates UV tube (18), controls operations of negative ion generator and UV tube.

This three in one electro-optical air sterilizer with ionizer uses UV tube to generate UV with wavelength of 253.7 nanometers, which verified by many scientific researches, is the most effective type of UV to kill bacteria, viruses and mildews in the air, and those increased negative ions by ion generator could promote biochemistry process in human body, lower our hormonal secretion on depression and fatigue. In this modem society, this electro-optical air sterilizer with ionizer can be used conveniently at families, hospitals, senior citizen centers, shopping malls, restaurants, office buildings, automobile manufacturers, council chambers, marketplaces, theaters, hotels, etc. Install this body at the entrance to separate air from outside, avoid hot and cold air exchange between indoor and outdoor, prevents dusts, contaminations, misquotes, flies, and odors. electro-optical air sterilizer with ionizer can be broadly applied in electronics, instrument medicine, food and various processing industries. To sum up, electro-optical air sterilizer with ionizer improves environments of our lives, habitations, medical treatments, work conditions, shopping etc.; provide us clean spaces in this constant nature demolishing modern society.

1. An air sterilizer, comprising:
a three-dimensional housing for actuating an assemblage having a plurality of air inlets and a plurality of air outlets with predetermined sharp, wherein the three-dimensional housing comprises:
at least one extractor fan mean horizontally inserted and engaged in said three-dimensional housing for mean of drawing air into said three-dimensional housing through said air inlet, through a ultraviolet radiation tube mean and out of said housing through said air outlet,
an air aggregator integrally provided between said extractor fan mean and said housing,
a ultraviolet radiation tube mean for generating ultraviolet rays to produce and increase the quantity of anions is integrally connected within said housing, said air inlet and air outlet are connected at both ends of said a ultraviolet radiation tube mean,
an electric circuit with a voltage transformer connected with an indicator light, a starter electrically connected via said electric circuit to said ultraviolet radiation tube,
a switch on said housing electrically connected via said electric circuit between said housing and said air drawing means, and a plug on an electric cord electrically connected to said electric circuit to engage with a wall socket to receive electricity therefrom;
a high negative voltage discharge/carbonated fiber is connected between said ultraviolet radiation tube and said air outlet.

2. An electronic circuit system comprising:
an anion generating circuit, and
an ultraviolet radiation tube generating 253.7 nm start-up circuit.

3. As recited in claim 2, wherein said electronic circuit system is characterized by a high negative voltage output volume as stated, i.e. within the range of 4.5 kv and 8.5 kv.

4. As recited in claim 2, wherein said electronic circuit system is characterized by a variable resistor functions of said anion generating circuit, i.e. to adjust high negative voltage output volume.

5. As recited in claim 2, wherein said electronic circuit system is characterized by a variable resistor functions of said ultraviolet radiation tube generating 253.7 nm start-up circuit, i.e. to adjust high negative voltage output volume.

6. As recited in claim 2, wherein said electronic circuit system is characterized by the fact that said air sterilizer works in two alternate modes in loop. Said two alternate modes in loops further comprises of:
first mode is to output high negative voltage intermittently (about 3 to 4 minutes between each interception) via said carbonated fiber discharge, said anions produced is then blown out through said extractor fan.
second mode is to ignite the extreme ultraviolet radiation intermittently (about 7 to 10 minutes between each interception) to eliminate the germs, viruses, funguses and other harmful airborne microorganisms or particles in the air. User may operate independently and continuously in mode 1 and/or mode 2.

7. As recited in claim 1, wherein there are fences at both front and rear portions of said air aggregator. Said fences keep said extreme ultraviolet radiations from radiating beyond said housing 1, so as to protect the eyes of the user.

8. An electronic circuit system as recited in claim 2, wherein an ultraviolet radioactive ray chamber produces 253.7 nanometers of waves to active an electronic circuit.

9. An electronic circuit system as recited in claim 2, wherein such electronic circuit is distinguished by its negative high voltage output value between 4.5 kv to 8.5 kv.

10. An electronic circuit system as recited in claim 2, wherein such electronic circuit is having variable resistor effect created by the anion-generating circuit is to adjust negative high voltage output value.

11. An electronic circuit system as recited in claim 2, wherein said air sterilizer can adapt both conventional indoor power supply and power generated by any transportation vehicles.

12. An air sterilizer as recited in claim 1, wherein said the UV tube and said dust filtration screen provide means of replacement.

13. An air sterilizer as recite in claim 1, wherein a three dimensional housing and inlet and outlet bars, which is mounted to the front plane of said body, and
an inlet frame grid is mounted to a front plane portion of frame grid, and
an ultraviolet radioactive ray chamber is located within a triangle in a center position of a filter frame grid, and
a contact chips at both front and rear portions of ultraviolet radioactive ray chamber are connecting to an external power via terminal box, while said ultraviolet radioactive ray chamber fastens a front frame grid and an rear frame grid in a half-circle, and
a carbon fibers are fastened within an extended pit of a front frame grid, and a front frame grid further comprises
a circuit board, electronic components, and external power line.
On a side face of a front frame grid, there is a rectangular hole-site which fastens said external power line. A switch is connected to a central section of said external power line.
In actual operation, this air sterilizer is usually installed at an exhaust inlet/outlet, within an external shield or outsides of an air conditioning device. The indoor air aspirated enters said front frame grid through said inlet frame grid, passes said central triangle in said filter frame grid, and is deprived of bacteria, viruses and molds by ultraviolet radioactive rays emitted from said ultraviolet radioactive ray chamber. Then fresh and clean air goes out of said rear frame grid through said outlet frame grid to enter said air conditioning device via said inlet, and discharges from said outlet of said air conditioning device.
